(19) United States
(12) Patent Application Publication
(10) Pub. No.: US 2008/0160610 A1
(43) Pub. Date: Jul. 3, 2008

(54) Methods for Stem Cell Expansion and Differentiation

(75) Inventors: Susan K. Nilsson, Victoria (AU); David Norman Haylock, Victoria (AU); Paul John Simmons, Victoria (AU)

Correspondence Address: BOZICEVIC, FIELD & FRANCIS LLP, 1900 UNIVERSITY AVENUE, SUITE 200, EAST PALO ALTO, CA, 94303, US

(73) Assignee: PETER MACCALLUM CANCER INSTITUTE, Melbourne, VIC (AU)

(21) Appl. No.: 11/568,557

(22) PCT Filed: May 3, 2005
(86) PCT No.: PCT/AU2005/000636

§ 371 Date: Aug. 24, 2007

(30) Foreign Application Priority Data
May 3, 2004 (AU) 2004902337

(51) Int. Cl. C12N 5/06

(52) U.S. Cl. 435/372; 435/377; 435/366; 435/405

(57) ABSTRACT

The present invention provides methods, culture media, and apparatus to produce useful amounts of specific cell populations ex vivo by the modulation of Opn and/or an active Opn fragment. The present invention provides ex vivo expanded populations of HSC for use in transplantation therapy and in clinical and research activities, such as drug screening, toxicity testing, and other research activities. Also provided are methods, devices and culture media are provided to inhibit Opn binding to HISC to promote the increased production of more differentiated cell populations.

### FIELD OF THE INVENTION

This invention relates generally to the ex vivo expansion, proliferation and differentiation of multipotential stem cell populations, methods for performing such methods, and products to facilitate the culture and use of clinically useful quantities of cell populations, both hematopoietic stem cells and cells of the hematopoietic lineage.

### BACKGROUND OF THE INVENTION

The bone marrow provides a unique environment for multipotential and committed cells. It contains both structural and humoral components that have yet to be successfully duplicated in culture. The marrow cavity itself is a network of thin-walled sinusoids lined with endothelial cells. Between the walls of bone are clusters of hematopoietic cells and fat cells constantly fed by mature blood cells entering through the endothelium. Differentiated cells ready to function within the circulatory system depart the cavity in a similar fashion.

Hematopoietic stem cells (HSC) are the most primitive cells of the hematopoietic lineage, and have the ability to give rise to all cells of the hematopoietic lineage (including HSC). HSC are known to reside in the bone marrow, but their specific niche within the bone marrow microenvironment is not currently defined. Previous studies have established that certain HSC progeny, the lineage-restricted clonogenic hematopoietic progenitor cells (HPC), conform to a well-defined spatial distribution across the axis of the femur with greatest numbers near the central longitudinal vein. Such observations foster the widely held belief that the distinct spatial organization exhibited by these various cell populations within the bone marrow is a manifestation of specific adhesive interactions occurring with the underlying stromal tissue. However, due to the rarity of HSC and the lack of a single, unique antigenic marker allowing their unambiguous identification in situ, it has not been possible to define the spatial distribution of HSC within the bone marrow.

Evidence now exists to suggest that hematopoiesis is localized to the bone marrow by developmentally regulated adhesive interactions between primitive HSC and the stromal cell mediated microenvironment. It is likely that the adhesive interactions in this microenvironment serve multiple functions, including homing and lodgment of HSC to the bone marrow during ontogeny or following transplantation, and participation in the direct regulation of their proliferation and differentiation.

Bone marrow transplantation is a useful treatment for a variety of hematological, autoimmune and malignant diseases, where there is a need to replenish hematopoietic cells of the bone marrow (via hematopoiesis) that have been depleted by treatments such as chemotherapy and radiotherapy. Current bone marrow transplantation therapies include the use of hematopoietic cells obtained from umbilical cord blood or from peripheral blood (either unmobilized or mobilized with agents such as G-CSF), as well as directly from the bone marrow.

A limitation in bone marrow transplantation is obtaining enough stem cells to restore hematopoiesis. Current therapies often rely the ex vivo manipulation of hematopoietic cells to expand primitive stem cells to a population suitable for transplantation. Moreover, whilst there is rapid regeneration to normal pre-transplantation levels in the number of hematopoietic progenitors and mature end cells following bone marrow transplantation, HSC numbers recover to only 5-10% of normal levels. This suggests that HSC are significantly restricted in their self-renewal behavior and hence in their ability to repopulate the host stem cell compartment. The available methodologies do not adequately address ex vivo HSC manipulation, and thus the cell populations used in clinical applications are limited by the number of cells that are able to be isolated from the donor. For example, due to the limited number of multipotential HPC in umbilical cord blood, cells from this source can only be used for transplantation in younger patients, and excludes the adult population in need of HSC transplantation therapies.

In addition to issues impacting upon therapeutic uses, there exists the problem of obtaining sufficient numbers of HSC for clinical studies, drug development, or research purposes. An understanding of HSC activity and behavior is tremendously important in improving the efficacy of therapies, and in determining the toxicity of various therapeutics. Isolation of normally occurring populations of stem or progenitor cells in adult tissues has been technically difficult and costly, due, in part, to the limited quantity of stem or progenitor cells found in blood or tissue, and the significant discomfort involved in obtaining bone marrow aspirates. In general, harvesting of stem or progenitor cells from alternative sources in adequate amounts for therapeutic and research purposes is generally laborious, the sources are limited due to the nature of the harvesting procedures, and the yield is low.

There is thus a need for methods and products for the ex vivo expansion of HSC products for use in therapeutic applications such as bone marrow transplantation. There is also a need for expansion and differentiation of cell populations to provide adequate numbers of specific cell populations for other applications, including research use, drug development and toxicity screening, and the production of mature, differentiated cell types. The present invention addresses this need.

### SUMMARY OF THE INVENTION

The present invention provides methods, culture media, and apparatus to produce useful amounts of specific cell populations ex vivo by the modulation of Opn and/or an active Opn fragment. The invention is based upon the finding that Opn binding to multipotential stem cells such as HSC from umbilical cord blood or HSC isolated from peripheral blood following mobilization inhibits overall cell proliferation from HSC, but enhances the specific expansion of the number of HSCs, leading to an increase in the HSC population of the culture. HSC cultured in the presence of Opn showed a marked reduction in the production of cells of the hematopoietic lineage, but displayed an increase in the number of multipotential HSC produced in the culture environment. Thus, Opn binding to HSC promotes expansion of the initial population of multipotential HSC, and in turn suppresses the proliferation and differentiation of HSC into progeny of the hematopoietic lineage.

An important aspect of this invention is that the binding of Opn (or an active Opn fragment) to HSC can be used to provide a cultured population of HSC that are self-renewable over a span of time, preferably at least three months, more preferably at least six months. Opn can be added as a factor to the media or provided as an immobilized form of Opn in a cell culture device to promote Opn binding and artificially recapitulate the HSC stromal-mediated microenvironmental niche for HSC expansion and maintenance of their multipotential state. Factors that potentiate Opn activity, such as the enzyme thrombin, can be added as an accessory factor to enhance Opn's activity in the culture.

It is a feature of the invention that introduction of Opn to a HSC population can be used to increase the number of cells useful for transplantation into a patient in need of such medical intervention, thus producing an expanded HSC population for transplantation. The ex vivo production of an expanded HSC population provides a transplantable cell population with increased numbers of multipotential cells, increasing the efficacy of the transplantation and allowing transplantation following the isolation of fewer HSC. Such a transplantable cell population can be produced following isolation of cells from bone marrow, from peripheral blood following mobilization through the use of an agent such as G-CSF, or from sources such as umbilical cord blood.

In a specific embodiment, the invention provides populations of HSC expanded from umbilical cord blood for transplantation to a patient in need thereof. HSC isolated from umbilical cord blood display certain characteristics that make them superior to cells derived from bone marrow. In particular, umbilical cord blood derived HSC and the progeny derived from cord blood do not appear to be as immunogenic as HSC from bone marrow, and thus show improved clinical outcomes in patients without a perfect HLA match. Currently, the use of such HSC is inhibited by the numbers of HSC that can be isolated from an umbilical source, which are not sufficient for engraftment in an adult. The possibility of using umbilical cord blood for transplantation in adults opens up the use of this cell source to a much wider patient population, and will allow many people who do not currently have an appropriate HLA matched donor to receive HSC transplantation therapy.

It is thus an object of the present invention to provide ex vivo expanded populations of HSC for transplantation therapy. The use of Opn in the ex vivo expansion process will allow specific expansion of HSC populations, resulting in greater transplantation efficiency.

It is one aspect of the invention that the ex vivo expansion of HSC can be undertaken with isolated cell populations enriched for HSC, e.g., cells isolated via identification of the CD34 surface marker.

The present invention also provides ex vivo expanded populations of HSC for use in clinical and research activities, such as drug screening, toxicity testing, and other research activities. The impact of therapeutic agents on hematopoiesis can be critical, especially in patients with severe pathologies, and in many cases this may compound the clinical problem. For example, anemia is a common side effect of therapeutic agents used to treat diseases including renal failure, congestive heart disease, and chronic obstructive pulmonary disease. Understanding the impact of these therapeutic agents on hematopoiesis may lead to improvement in these products to eliminate this side effect in such patient populations, resulting in the development of agents that provide better clinical outcomes. The invention envisions the use of HSC expanded through use of Opn for these and other related activities.

It is thus one object of the invention to provide a population of ex vivo expanded HSC for drug screening and optimization of therapeutically active agents.

It is another object of the invention to provide a population of ex vivo expanded HSC for toxicity testing. Toxicity testing of therapeutic agents will allow identification of an adverse impact on hematopoiesis without requiring testing in a patient population. Testing of drugs on human cell populations such as HSC can be used to provide evidence of safety of a therapeutic agent to the regulatory bodies such as the U.S. Food and Drug Administration.

In a specific embodiment, the invention provides cell culture media containing sufficient levels of Opn to promote Opn binding to HSC in the media. This enriched media promotes specific expansion of the HSC while suppressing additional proliferation and differentiation of more differentiated cell types. In a more particular embodiment, this medium may be enhanced by the addition of thrombin, which potentiates Opn binding to HSC via production of an active Opn fragment, e.g., through production of an Opn fragment with an epitope more accessible to HSC binding. The media may be used in any conventional cell growth device, including flasks, bioreactors and the like. This media may contain other important factors, including cytokines, growth factors, and factors that enhance Opn activity (e.g., thrombin).

In yet another embodiment, the invention provides a culture device wherein Opn or an active Opn fragment is immobilized to a surface of a culture flask, bead, or other surface (such as the surface of a bioreactor), and HSC are exposed to the Opn/immobilizing surface to enhance HSC production and prevent proliferation and differentiation of the HSC progeny. This culture device uses Opn binding to promote growth and expansion of the HSC population, maintaining the multipotentiality of both the parent HSC and the multipotential progeny HSC. This includes bioreactor culture devices on which Opn is immobilized on the surface. The surface may also comprise other immobilized molecules that, in conjunction with Opn, artificially recapitulate the HSC stromal-mediated microenvironmental niche.

In a separate embodiment of the invention, methods, devices and culture media are provided to inhibit Opn binding to HSC to promote the increased production of more differentiated cell populations. These methods result in an increased number of cells produced in the hematopoietic lineage, which can subsequently be used in other specific therapeutic applications requiring the introduction of cells from the hematopoietic lineage.

It is thus an object of the present invention to provide ex vivo expanded populations of HSC for transplantation therapy.

It is an object of the invention to inhibit or prevent Opn binding to HSC to increase overall proliferation and differentiation of HSC populations, and to produce and isolate more mature cell populations from the hematopoietic lineage. This can be an active inhibition, if Opn is present, or a passive inhibition through providing a culturing environment devoid of any Opn. Active inhibition may be direct or indirect, i.e. act directly on the Opn molecule, or inhibit the activity of a molecule required for Opn activity in the culture environment.

The invention thus provides cell populations for therapeutic treatment of patients. The introduction of more differentiated cells of the hematopoietic system can include populations of any cell of the hematopoietic lineage, including cells from the myeloerythroid (red blood cells, granulocytes, and monocytes), megakaryocyte (platelets) and lymphoid (T-cells, B-cells, and natural killer cells) lineages. The cell population introduced to the patient will depend upon the pathology, and the cells can be introduced in an isolated population or in a mixed population, e.g., a cell population that clinically approximates whole blood.

In one aspect, the cell populations are isolated to one specific cell type, e.g., red blood cells. In another aspect, the cell population may be a heterogeneous population of HSC progeny.

The invention also features cell culture media and devices for the production of differentiated hematopoietic cell populations. In a specific embodiment, the invention provides cell culture media containing sufficient levels of one or more agents that block Opn binding to HSC. This media will allow maintenance of HSC levels while promoting proliferation and differentiation of more mature cell types in the hematopoietic lineage. In a more particular embodiment, this medium may be enhanced by the addition of an agent that inhibits thrombin, which as describe can potentiates Opn binding to HSC via production of an active Opn fragment. The media may be used in any conventional cell growth device, including flasks, bioreactors and the like.

In one specific embodiment of the invention, cell production is undertaken in a bioreactor designed for producing clinically useful quantities of mature cells of the hematopoietic lineage. Such a system would require the decreasing Opn binding to HSC to promote increased proliferation of the HSC into adequate numbers of differentiated cells. In a specific aspect, the selection system is comprised of sequential system providing Opn binding of cultured HSCs, with Opn or an active Opn fragment initially provided to the cells to promote expansion of the HSC “culture” population, followed by inhibition of Opn binding to promote the increased proliferation and differentiation of cells.

The invention also features a method for activating quiescent HSC to divide by exposing such cells to Opn to promote uptake of an agent (e.g., a small molecule, protein, oligonucleotide, vector or other gene delivery device) to promote or modulate gene expression or protein production in a cell.

Accordingly, in a related aspect, quiescent stem cells are activated in the presence of Opn or an active Opn fragments, including activation with Opn in the presence of thrombin, and cultured with an active agent or delivery vector.

These and other objects, advantages and features of the present invention will become apparent to those persons skilled in the art upon reading the details of the structure of the device, formulation of compositions and methods of use, as more fully set forth below.

### BRIEF DESCRIPTION OF THE DRAWINGS

So that the manner in which the above recited features, advantages and objects of the present invention are attained and can be understood in detail, a more particular description of the invention, briefly summarized above, may be had by reference to the embodiments that are illustrated in the appended examples and drawings. It is to be noted, however, that the appended examples and drawings illustrate only certain embodiments of this invention and are therefore not to be considered limiting of its scope, for the present invention may admit to other equally effective embodiments.

FIG. 1 is a bar graph illustrating the spatial distribution of HSC isolated from either CD44^{-/-} or C57B6 mice upon transplantation without ablation.

FIG. 2 illustrates donor reconstitution following a transplant of wild type HSC into different hematopoietic microenvironments. HSC=donor hematopoietic stem cells; HM=recipient microenvironment.

FIG. 3 is a bar graph illustrating adhesion of murine HSC to Opn. CD44 binding is in the presence of EDTA, while VLA4 binding is in the presence of MnCl₂.

FIG. 4 is a bar graph illustrating that Opn binding to HSC inhibits proliferation of hematopoietic progeny. Hematopoietic progenitors produced were measured per 500 CD34⁺ CB HSC seeded in serum-free culture for 4 days. HGF=hematopoietic growth factors.

FIG. 5 is a bar graph illustrating the cell cycle history of Opn−/− and wild type controls following 4 weeks of BrdU. The graph measures percentage of Lin-Sca+Kit+ cells cycling following 4 weeks continuous BrdU. This graph illustrates the ability of Opn to promote HSC expansion.

FIG. 6 is a bar graph demonstrating that the absence of Opn in the stroma inhibits the migration of HSC into the stroma.

FIG. 7 is a bar graph demonstrating that immobilized Opn prevents HSC chemotaxing to an SDF-1 gradient.

### DETAILED DESCRIPTION

Before the present devices, cells and methods of cell production are described, it is to be understood that this invention is not limited to the particular methodology, products, apparatus and factors described, as such methods, apparatus and formulations may, of course, vary. It is also to be understood that the terminology used herein is for the purpose of describing particular embodiments only, and is not intended to limit the scope of the present invention which will be limited only by appended claims.

It must be noted that as used herein and in the appended claims, the singular forms “a,” “and,” and “the” include plural references unless the context clearly dictates otherwise. Thus, for example, reference to “a factor” refers to one or mixtures of factors, and reference to “the method of production” includes reference to equivalent steps and methods known to those skilled in the art, and so forth.

Unless defined otherwise, all technical and scientific terms used herein have the same meaning as commonly understood by one of ordinary skill in the art to which this invention belongs. All publications mentioned herein are incorporated herein by reference for the purpose of describing and disclosing devices, formulations and methodologies which are described in the publication and which might be used in connection with the presently described invention.

In the following description, numerous specific details are set forth to provide a more thorough understanding of the present invention. However, it will be apparent to one of skill in the art that the present invention may be practiced without one or more of these specific details. In other instances, well-known features and procedures well known to those skilled in the art have not been described in order to avoid obscuring the invention. For example, additional description of apparatus, methods, cell populations and appropriate factors that could be employed for the methods of expansion and differentiation described herein include those described in U.S. Pat Nos. 5,399,493; 5,472,867; 5,635,386; 5,635,388; 5,646,043; 5,674,750; 5,925,567; 6,403,559; 6,455,306; 6,258,597; and 6,280,718.

Generally, conventional methods of cell culture, stem cell biology, and recombinant DNA techniques within the skill of the art are employed in the present invention. Such techniques are explained fully in the literature, see, e.g., Maniatis, Fritsch & Sambrook, Molecular Cloning: A Laboratory Manual (1982); Sambrook, Russell and Sambrook, Molecular Cloning: A Laboratory Manual (2001); Harlow, Lane and Harlow, Using Antibodies: A Laboratory Manual : Portable Protocol NO. I, Cold Spring Harbor Laboratory (1998); and Harlow and Lane, Antibodies: A Laboratory Manual, Cold Spring Harbor Laboratory; (1988).

Although the present invention is described primarily with reference to HSC, it is also envisioned that Opn and its cell surface interactions may play a role in the regulation of other stem cell populations (including known stem cells such as mesenchymal stem cells or other yet unidentified stem cells) that are involved in lodgment in a microenvironmental niche. The invention is intended to cover these Opn modulation in these stem cell populations as well as in HSC.

Definitions

The term “active Opn fragment” as used herein includes active Opn fragments maintaining the HSC expanding activity of Opn in the described methods. This includes cleavage products of Opn, including but not limited to cleavage products produces by the interaction of Opn with the enzyme thrombin.

The term “blood cells” is intended to include erythrocytes (red blood cells), reticulocytes, megakaryocytes, eosinophils, neutrophils, basophils, platelets, monocytes, macrophages, granulocytes and cells of the lymphoid lineage. For the purpose of transfusion of mature cell populations into patients, erythrocytes, granulocytes and platelets are particularly valuable. The phrase “clinically useful quantities (or amounts) of blood cells” is intended to mean quantities of blood cells of whatever specific population is sufficient for transfusion into human patients to treat a clinical condition.

The terms “Hematopoietic stem cell”, “HSC” and the like are used herein to mean a stem cell having (1) the ability to give rise to progeny in all defined hematopoietic lineages, and (2) stem cells capable of fully reconstituting a seriously immunocompromised host in all blood cell types and their progeny, including the multipotential hematopoietic stem cell, by self-renewal. A multipotential hematopoietic stem cell may be identified by expression of the cell surface marker CD34⁺.

The term “multipotential” as used herein refers to the ability to produce any cell of the hematopoietic lineage.

The terms “Osteopontin”, “Opn” and like terms used herein refer to a form of the protein osteopontin or a fragment thereof capable of performing its intended function both in vivo, e.g., a form capable of influencing early bone matrix organization, as well as ex vivo in the methods of the invention. Opn is a phosphorylated acidic glycoprotein that exists as an immobilized ECM in mineralized tissues, synthesized primarily by cells of the bone lineage, and as a cytokine.

Examples of osteopontin forms useful in the invention are: a phosphorylated osteopontin, e.g., an osteopontin having about 6 to about 12 phosphates per mol of protein, preferably, an osteopontin phosphorylated at one or more of the following amino acids selected from the group consisting of Ser26, Ser27, Ser63, Ser76, Ser78, Ser81, Ser99, Ser102, Ser105, Ser108, Ser117, Thr138, and/or Thr152. The forms envisioned for use in the present invention include a recombinant osteopontin, e.g., a human or murine recombinant osteopontin, and a naturally occurring isolated osteopontin, e.g., the naturally occurring osteopontin isolated from a human source.

Throughout the description and claims of this specification the word “comprise”, and variations of the word such as “comprising” and “comprises”, is not intended to exclude other additives or components or integers or steps.

Homing and Lodgment of HSC in the BM: The Role of Cell Adhesion Molecules (CAMs).

The reestablishment of hematopoiesis by intravenously infused bone marrow requires several coordinated events including homing, migration and lodgment of HPC within the bone marrow microenvironment. The initial event, homing, is defined as the specific recruitment of circulating HSC to the bone marrow and involves the selective recognition by HSC of the microvascular endothelium of the bone marrow and trans-endothelial cell migration into the extravascular hematopoietic space. In contrast, lodgment encompasses events following extravasation and is defined as the selective migration of cells to suitable microenvironmental niches in bone marrow extravascular hematopoietic space.

HSC homing involves a similar cascade of CAMs to those which participate in the extravasation of mature leukocytes into tissues (Butcher, E. C., Cell, 1991. 67. p. 1033-6). HSC exhibit a broad repertoire of CAMs including various members of the integrin, sialomucin, Ig superfamily and CD44 families (reviewed in Simmons, P. J., et al., Leukemia and Lymphoma, 1994. 12. p. 353-363; Simmons, P. J., J.-P. Levesque, and A. Zannettino, Bailliere's Clinical Haematology, 1997. 10. p. 485-505). Current data suggest key roles for the sialomucin receptor for P-selectin, PSGL-1 (Frenette, P. S., et al., Proceedings of the National Academy of Sciences, 1998. 95. p. 14423-14428), the β₁ integrin VLA-4 (Papayannopoulou, T., et al., Proc Natl Acad Sci USA, 1995. 92. p. 9647-9651) and the receptor for SDF-1, CXCR4 (Peled, A., et al., Science, 1999. 283. p. 845-8) in the homing of HSC to the bone marrow. In contrast, very little is known about the molecules that influence the site of HSC lodgment following homing to the bone marrow.

The present invention is based in part on the unexpected finding that Opn is necessary for lodgment in the endosteal space via interactions with CD-44. The importance of CD-44, both on the HSC cell surface and in the hematopoietic microenvironment, and its interaction with Opn suggests methods for recapitulating the hematopoietic microenvironmetal niche.

Upon further investigation by the inventor, Opn was also found to play an integral role in HSC lodgment, regulation and proliferation, and in particular on the ability of HSC to expand into additional HSC or, alternatively, to proliferate into more differentiated cells of the hematopoietic lineage. The present invention is based in large part upon the scientific observation related to Opn's activity in HSC regulation, and the methods, culture media, and devices described take advantage of Opn's unique properties in stem cell regulation, expansion, and proliferation.

The role of Opn is also not completely dependent upon its interaction with CD-44, and in fact involves SDF-1 interaction to allow migration of HSC to the stroma.

Expansion of HSC in Ex Vivo Culture: Cell Sources

HSC may be isolated from any known human source of stem cells, including bone marrow, both adult and fetal, mobilized peripheral blood, and umbilical cord blood. Initially, bone marrow cells may be obtained from a source of bone marrow, including ilium (e.g., from the hip bone via the iliac crest), tibia, femora, spine, or other bone cavities. Other sources of stem cells include embryonic yolk sac, fetal liver, and fetal spleen. The HSC sourced for use in the methods of the invention can comprise a heterogeneous population of cells including a combination of multipotential HSC, immunocompotent cells and stromal cells including fibroblast and endothelial cells.

Umbilical cord blood is comparable to bone marrow as a source of hematopoietic stem cells and progenitors (Broxmeyer et al., 1992; Mayani et al., 1993). In contrast to bone marrow, cord blood is more readily available on a regular basis.

Methods for mobilizing stem cells into the peripheral blood are known in the art and generally involve treatment with chemotherapeutic drugs, e.g., cytoxan, cyclophosphamide, VP-16, and cytokines such as GM-CSF, G-CSF, or IL-3, or combinations thereof. Typically, apheresis for total white cells begins when the total white cell count reaches 500-2000 cells/μl and the platelet count reaches 50,000/μl. Daily leukapheris samples may be monitored for the presence of CD34⁺ and/or Thy-1⁺ cells to determine the peak of stem cell mobilization and, hence, the optimal time for harvesting peripheral blood stem cells.

Enrichment of HSC from Sourced Cells

Binding of Opn or an active Opn fragment to HSC provides a novel and potent means of improving various ex vivo manipulations such as ex vivo expansion of stem cells and genetic manipulation of stem cells. The HSC used in such a device preferably are isolated HSC populations, although it is intended that the methods, media and devices of the invention can also be used for ex vivo expansion of HSC in heterogeneous cell populations such as adult human bone marrow or human umbilical cord blood cells.

An example of an enriched HSC population is a population of cells selected by expression of the CD34⁺ marker. In LTCIC assays, a population enriched in CD34⁺ cells will typically have an LTCIC frequency in the range of 1/50 to 1/500, more usually in the range of 1/50 to 1/200. Preferably, the HSC population will be more highly enriched for HSC than that provided by a population selected on the basis of CD34⁺ expression alone. By use of various techniques described more fully below, a highly enriched HSC population may be obtained. A highly enriched HSC population will typically have an LTCIC frequency in the range of ⅕ to 1/100, more usually in the range of 1/10 to 1/50. Preferably, it will have an LTCIC frequency of at least 1/50. Exemplary of a highly enriched HSC population is a population having the CD34⁺ Lin⁻ or CD34⁺ Thy-I⁺ Lin⁻ phenotype as described in U.S. Pat. No. 5,061,620 incorporated herein by reference to disclose and describe such cells. A population of this phenotype will typically have an average LTCIC frequency of approximately 1/20 (Murray et al., Enrichment of Human Hematopoietic Stem Cell Activity in the CD34⁺ Thy-1⁺ Lin− Subpopulation from Mobilized Peripheral Blood, Blood, vol. 85, No. 2, pp. 368-378 (1995); Lansdorp et al. (1993) J. Exp. ed. 177:1331). LTCIC frequencies are known to correlate with CAFC frequencies (Reading et al., Proceedings of ISEH Meeting 1994, Abstract, Exp. Hematol., vol. 22:786, 406, (1994).

Various techniques may be employed to separate the cells by initially removing cells of dedicated lineage (“lineage-committed” cells). Monoclonal antibodies are particularly useful for identifying markers associated with particular cell lineages and/or stages of differentiation. The antibodies may be attached to a solid support to allow for crude separation. The separation techniques employed should maximize the viability of the fraction to be collected.

The use of separation techniques include those based on differences in physical (density gradient centrifugation and counter-flow centrifugal elutriation), cell surface (lectin and antibody affinity), and vital staining properties (mitochondria-binding dye rhodamine 123 and DNA-binding dye Hoechst 33342). Procedures for separation may include magnetic separation, using antibody-coated magnetic beads, affinity chromatography, cytotoxic agents joined to a monoclonal antibody or used in conjunction with a monoclonal antibody, including complement and cytotoxins, and “panning” with antibody attached to a solid matrix or any other convenient technique. Techniques providing accurate separation include flow cytometry which can have varying degrees of sophistication, e.g., a plurality of color channels, low angle and obtuse light scattering detecting channels, impedance channels, etc.

A large proportion of the differentiated cells may be removed by initially using a relatively crude separation, where major cell population lineages of the hematopoietic system, such as lymphocytic and myelomonocytic, are removed, as well as lymphocytic populations, such as megakaryocytic, mast cells, eosinophils and basophils. Usually, at least about 70 to 90 percent of the hematopoietic cells will be removed.

Concomitantly or subsequent to a gross separation providing for positive selection, e.g. using the CD34 marker, a negative selection may be carried out, where antibodies to lineage-specific markers present on dedicated cells are employed. For the most part, these markers include CD2⁻, CD3⁻, CD7⁻, CD8⁻, CD10⁻, CD14⁻, CD15⁻, CD16⁻, CD19⁻, CD20⁻, CD33⁻, CD38⁻, CD71⁻, HLA-DR⁻, and glycophorin A; preferably including at least CD2⁻, CD14⁻, CD15⁻, CD16⁻, CD19⁻ and glycophorin A; and normally including at least CD14⁻ and CD15⁻. As used herein, Lin⁻ refers to a cell population lacking at least one lineage specific marker. The hematopoietic cell composition substantially depleted of dedicated cells may be further separated using selection for Thy-1⁺ and/or Rho123^{lo}, whereby a highly enriched HSC population is achieved.

The purified HSC have low side scatter and low to medium forward scatter profiles by FACS analysis. Cytospin preparations show the enriched HSC to have a size between mature lymphoid cells and mature granulocytes. Cells may be selected based on light-scatter properties as well as their expression of various cell surface antigens.

Cells can be initially separated by a coarse separation, followed by a fine separation, with positive selection of a marker associated with HSC and negative selection for markers associated with lineage committed cells. Compositions highly enriched in HSC may be achieved in this manner. The desired stem cells are exemplified by a population with the CD34⁺ Thy-1⁺ Lin⁻ phenotype, and are characterized by being able to be maintained in culture for extended periods of time, being capable of selection and transfer to secondary and higher order cultures, and being capable of differentiating into the various lymphocytic and myelomonocytic lineages, particularly B- and T-lymphocytes, monocytes, macrophages, neutrophils, erythrocytes and the like.

Culture Methods and Devices for Expansion of HSC Populations

Opn or a fragment thereof can be added to the media to promote Opn binding to HSC and artificially recapitulate the HSC stromal-mediated microenvironmental niche. The specific HSC expansion media can be used to establish and maintain a multipotential HSC population for various uses. In a specific embodiment, the culture media also contains thrombin to further enhance Opn binding to HSC.

Alternatively, Opn or a fragment thereof can be immobilized to a surface of a culture flask, bead, or other surface of a culture device (such as the surface of a bioreactor), and the HSC exposed to the Opn/immobilizing surface. HSC will bind the appropriate Opn or active Opn fragment in or on the culture device, which will have two major effects: 1) the Opn or active Opn fragments will immobilize the cell on the surface in the culture system and 2) the Opn or active Opn fragments will promote expansion of the multipotential HSC population.

Immobilized Opn can be used in conjunction with other immobilized proteins that bind to HSC (such as agents that bind to angiotensin converting enzyme (ACE), CD59, CD34 and/or Thy-1) in either the culture media or alternatively immobilized on the culture device to artificially recapitulate elements of the HSC microenvironmental niche. Upon cell division of the HSC, the multipotential HSC progeny produced will also bind to Opn, thus expanding the number of immobilized cells in the culture system.

Cells not expressing the appropriate cell adhesion molecules for Opn binding will not become immobilized, and thus can be removed from the culture system. For example, where Opn is immobilized in a flow through bioreactor, any HSC progeny not binding to Opn would be separated from the HSC culture during the flow through of the culture media. Thus, differentiating cells lacking the cell surface receptors for Opn binding can eluted or otherwise separated from the bound cells. This will allow not only expansion of the primordial HSC population, but will also promote greater homogeneity of this population through a de facto Opn selection process.

In one embodiment, the invention provides an HSC production device, i.e. a culture device for ex vivo expansion of multipotential HSC populations. This production device will deliver Opn to an HSC population in either immobilized for or via media introduced to the culture device. Preferably, the HSC population has been isolated from its starting material using one or a combination of cell surface markers, e.g., CD34 or angiotensin converting enzyme (ACE), prior to introduction of the HSC to the culture device. It is envisaged, however, that the HSC may be present in a heterogeneous cell population prior to introduction to the device, with the device having the ability to isolate the relevant HSC population based on other immobilized molecules that preferentially bind to the HSCs. Such heterogeneous populations include HSC present in adult human bone marrow or human umbilical cord blood cells.

The bioreactors that may be used in the present invention provide a culture process that can deliver medium and oxygenation at controlled concentrations and rates that mimic nutrient concentrations and rates in vivo. Bioreactors have been available commercially for many years and employ a variety of types of culture technologies. Once operational, bioreactors provide automatically regulated medium flow, oxygen delivery, and temperature and pH controls, and they generally allow for production of large numbers of cells. The most sophisticated bioreactors allow for set-up, growth, selection and harvest procedures that involve minimal manual labor requirements and open processing steps. Such bioreactors optimally are designed for use with a homogeneous cell mixture such as the bound HSC populations contemplated by the present invention.

Of the different bioreactors used for mammalian cell culture, many have been designed to allow for the production of high density cultures of a single cell type and as such find use in the present invention. Typical application of these high density systems is to produce, as the end-product, a conditioned medium produced by the cells. This is the case, for example, with hybridoma production of monoclonal antibodies and with packaging cell lines for viral vector production. One aspect of the invention is thus the production of conditioned HSC media where the end-product is the HSC conditioned media.

Suitable bioreactors for use in the present invention include but are not limited to those described in U.S. Pat. No. 5,763,194 to Slowiaczek, et al., particularly for use as the culture bioreactor; and those described in U.S. Pat. Nos. 5,985,653 and 6,238,908 to Armstrong, et al., U.S. Pat. No. 5,512,480 to Sandstrom, et al., and U.S. Pat. Nos. 5,459,069, 5,763,266, 5,888,807 and 5,688,687 to Palsson, et al., particularly for use as the proliferation and differentiation bioreactors of the present invention.

Attachment of Opn to a Culture Device Surface

Non-covalent attachment is known in the art and includes, but is not limited to, attachment via a divalent ion bridge, e.g., a Ca++, Mg++ or Mn++ bridge; attachment via absorption of Opn or a fragment thereof to the material; attachment via plasma spraying or coat drying of a polyamine, e.g., polylysine, polyarginine, spermine, spermidine or cadaverin, onto the material; attachment via a second polypeptide, e.g., fibronectin or collagen, coated onto the material; or attachment via a bifunctional crosslinker, e.g., N-Hydroxysulfosuccinimidyl-4-azidosalicylic acid (Sulfo-NHS-ASA), Sulfosuccinimidyl(4-azidosalicylamido) hexanoate (Sulfo-NHS-LC-ASA), N-γ-maleimidobutyryloxysuccinimide ester (GMBS), N-γ-maleimidobutyryloxysulfosuccinimide ester (Sulfo-GMBS), 4-Succinimidyloxycarbonyl-methyl-α-(2-pyridyldithio)-toluene (SMPT), Sulfosuccinimidyl 6[α-methyl-α(2-pyridyldithio)toluamido]hexanoate (Sulfo-LC-SMPT), N-Succinimidyl-3-(2-pyridyldithio)propionate (SPDP), Succinimidyl 6-[3-(2-pyridyidithio)propionamido]hexanoate (LC-SPDP), Sulfosuccinimidyl 6-[3-(2-pyridyldithio)propionamido]hexanoate (Sulfo-LC-SPDP), Succinimidyl 4-(N-maleimidomethyl)cyclohexane-1-carboxylate (SMCC), Sulfosuccinimidyl 4-(N-maleimidomethyl)cyclohexane-1-carboxylate (Sulfo-SMCC), m-Maleimidobenzoyl-N-hydroxysuccinimide ester (MBS), m-Maleimidobenzoyl-N-hydroxysulfosuccinimide ester (Sulfo MBS), N-Succinimidy(4-iodoacetyl)amino benzoate (SIAB), Sulfosuccinimidyl(4-iodoacetyl)amino benzoate (Sulfo-SIAB), Succinimidyl 4-(ρ-maleimidophenyl) butyrate (SMPB), Sulfosuccinimidyl 4(ρ-maleimidophenyl) butyrate (Sulfo-SMPB), or Azidobenzoyl hydrazide (ABH), to the material. In other embodiments Opn or an active fragment of Opn is attached to the material via an electrostatic interaction.

Alternatively, the Opn can be attached to a surface via non-covalent attachment, as described above, further including a glycosaminoglycan. Based on the interaction between Opn, CD44 and hyaluronic acid, the preferred glycosaminoglycan is hyaluronic acid, and more preferably hyaluronic acid greater than a disaccharide. In one embodiment the hyaluronic acid has a molecular weight range of less than 100 kDa, more preferably between about 20 to about 100 kDa, e.g., between about 50-100, 70-100, or 30-80 kDa.

Culturing Media and Devices for Promoting Cell Proliferation and Differentiation

When HSCs divide, some if not all the divisions are asymmetric. In asymmetric division, an initial HSC, divides to produce a daughter HSC and a more differentiated progeny cell. Asymmetric division leads to a steady state HSC population, generating a population of progeny cells to be used with or without further differentiation. One aspect of the present invention is based on the finding that inhibition of Opn in HSC culture promotes overall cell proliferation. Inhibition of Opn in the present invention can be used to exploit the asymmetric process by increasing the rate of asymmetric division in a culture system.

The bioreactor and culture conditions used to proliferate the more differentiated cells will vary depending on the ultimate mature cell product desired. Several “classic” bioreactors are known in the art and may be used, including bioreactor as as described in U.S. Pat. Nos. 5,985,653 and 6,238,908 to Armstrong, et al., U.S. Pat. No. 5,512,480 to Sandstrom, et al., and U.S. Pat. Nos. 5,459,069, 5,763,266, 5,888,807 and 5,688,687 to Palsson, et al.

The differentiated cell populations following Opn-blocking proliferation may be hemangioblasts, or other uncommitted common precursors of mature, completely differentiated blood cells. Hemangioblasts are stable, non-transient cells that are present in both newborn infants and adults and have been isolated from cord blood. Hemangioblasts can be proliferated in a first step followed by further proliferation to the desired blood cell. The further differentiated cells can be distinguished from primordial cells by cell surface markers, and the desired cell type can be identified or isolated based on such markers. For example, LIN-HSC lack several markers associated with lineage committed cells. Lineage committed markers include those associated with T cells (such as CD2, 3, 4 and 8), B cells (such as CD10, 19 and 20), myeloid cells (such as CD14, 15, 16 and 33), natural killer (“NK”) cells (such as CD2, 16 and 56), RBC (such as glycophorin A), megakaryocytes (CD41), or other markers such as CD38, CD71, and HLA-DR. Populations highly enriched in HSC and methods for obtaining them are described in PCT/US94/09760; PCT/US94/08574 and PCT/US94/10501.

Other culture conditions, such as medium components, O₂ concentration, differentiation factors, pH, temperature, etc., as well as the bioreactor employed, will vary depending on the desired cell population to be differentiated and the desired differentiated cell type, but will differ primarily in the cytokine(s) used to supplement the differentiation medium. The maturation process into a specific lineage can be modulated by a complex network of regulatory factors. Such factors include cytokines that are used at a concentration from about 0.1 ng/mL to about 500 ng/mL, more usually 10 ng/mL to 100 ng/mL. Suitable cytokines include but are not limited to c-kit ligand (KL) (also called steel factor (Stl), mast cell growth factor (MGF), and stem cell growth factor (SCGF)), macrophage colony stimulating factor (MCSF), IL-1α, IL-2, IL-3, IL-4, IL-5, IL-6, IL-11, G-CSF, GM-CSF, MIP-1, LIF, c-mpl ligand/thrombopoietin, erythropoietin, and flk2/flk3 ligand. The differentiation culture conditions will include at least two of the cytokines listed above, and may include several.

For example, if red blood cells are the desired mature blood product, at least erythropoietin will be added to the culture medium, and preferably SCGF, IL-1, IL-3, IL-6 and GMCSF all will be added to the culture medium, possibly with erythropoietin added later as a terminal differentiating factor. If platelets are the desired mature blood product, preferably SCGF, IL-1, IL-3, GMSCF and IL-11 will be added to the culture medium. For example, the path for the differentiation of T cells requires that the cell population be differentiated with IL-1 and IL-6, followed by differentiation with IL-1, IL-2 and IL-7, followed by differentiation with IL-2 and IL-4.

Alternatively to directing differentiation to a single cell type, the final product could be a mixed population and the cells could be separated using current cell separation techniques and procedures.

Inhibition of Opn binding to HSC also has utility in providing cell populations for applications such as research, screening for compounds or agents that alter HSC function or viability, toxicity testing of pharmaceutical agents and the like. Providing an HSC starting culture, and selectively enhancing proliferation of more mature cell types via inhibition of Opn binding to HSC, will allow not only an increase in HSC proliferation but specifically promote production of the more differentiated progeny.

Thus, in one embodiment, the invention provides media for HSC proliferation and differentiation containing one or more agents that inhibit Opn. The inhibition of Opn may be provided either in a single culture system, or in sequential culture systems (i.e., sequential bioreactors with different media). This is particularly useful if the culture system involves sequential culture conditions.

For example, to maximize the number of differentiated progeny produced, it may be desirable to first expand the HSC population via Opn binding, (with Opn provided immobilized in the culture setting or provided to the culture setting via media containing Opn) followed by inhibition of Opn to accelerate proliferation and differentiation of the more mature hematopoietic progeny.

Although a single Opn inhibitor may be used in the methods of the invention, in one embodiment it would be preferable to use multiple agents, (e.g., multiple antibodies to various Opn epitopes) to ensure the inhibition of Opn in the culture system and/or media, especially as Opn is known to bind to multiple cell adhesion molecules. The Opn inhibitory molecules contained in the media can be replenished by media perfusion. Alternatively, the Opn inhibitory molecules may be added separately, without media perfusion, as a concentrated solution through separate means in the culture system (e.g., into inlet ports in a bioreactor). When a binding agent is added without perfusion, it will typically be added as a 10-100×solution in an amount equal to one-tenth to 1/100 of the volume in the culture system, although it will of course depend on the actual affinity of the particular agent or agents to Opn.

In an exemplary embodiment, Opn binding and/or inhibition is used in the production of blood cells. Once differentiated, selection for the desired blood cell type can be performed by looking for cell surface markers. For example, T cells are known to have the markers CD2, 3, 4 and 8; B cells have CD10, 19 and 20; myeloid cells are positive for CD14, 15, 16 and 33; natural killer (“NK”) cells are positive for CD2, 16 and 56; red blood cells are positive for glycophorin A; megakaryocytes have CD41; and mast cells, eosinophils and basophils are known to have markers such as CD38, CD71, and HLA-DR.

Once produced, the blood cells may also be preserved for future use. Preservation of blood cells can be accomplished by any method known in the art. For example, general protocols for the preservation and cryopreservation of biological products such as blood cells are disclosed in U.S. Pat. Nos. 6,194,136 and 5,364,756 to Livesey, et al.; and 6,602,718 to Augello, et al. In addition, solutions and methods for the preservation of red blood cells are disclosed in U.S. Pat. No. 4,386,069 to Estep, and preservation of platelets is disclosed in U.S. Pat. Nos. 5,622,867, 5,919614, and 6,211,669 to Livesey, et al., as well as recent reports regarding new methods from HyperBaric Systems, Inc. and Human Biosystems, Inc.

It is envisioned that the cells produced using the methods of the invention can be used therapeutically to treat various blood disorders. The use of Opn in the culturing system will promote the expansion of the HSC into therapeutically relevant amounts of cells.

In a specific embodiment, the cells produced are erythrocytes (red blood cells). The major function of red blood cells is to transport oxygen to tissues of the body. Minor functions include the transportation of nutrients, intercellular messages and cytokines, and the absorption of cellular metabolites. Anemia, or a loss of red blood cells or red blood cell capacity, can be grossly defined as a reduction in the ability of blood to transport oxygen and may be acute or chronic. Chronic blood loss may be caused by extrinsic red blood cell abnormalities, intrinsic abnormalities or impaired production of red blood cells. Extrinsic or extra-corpuscular abnormalities include antibody-mediated disorders such as transfusion reactions and erythroblastosis, mechanical trauma to red cells such as micro-angiopathic hemolytic anemias, thrombotic thrombocytopenic purpura and disseminated intravascular coagulation. In addition, infections by parasites such as Plasmodium, chemical injuries from, for example, lead poisoning, and sequestration in the mononuclear system such as by hypersplenism can provoke red blood cell disorders.

Some of the more common diseases of red cell production include aplastic anemia, hypoplastic anemia, pure red cell aplasia and anemia associated with renal failure or endocrine disorders. Disturbances of the proliferation and differentiation of erythroblasts include defects in DNA synthesis such as impaired utilization of cyanocobalamin or folic acid and the megaloblastic anemias, defects in heme or globin synthesis, and anemias of unknown origins such as sideroblastic anemia, anemia associated with chronic infections such as malaria, trypanosomiasis, HIV, hepatitis virus or other viruses, and myelophthisic anemias caused by marrow deficiencies.

Promotion of Agent and Vector Uptake Through HSC Expansion

In a specific therapeutic aspect of the invention, hematopoietic cells are removed from a subject, transduced ex vivo, and the modified cells returned to the subject. The modified HSC and their progeny will express the desired gene product in vivo, thus providing sustained therapeutic benefit.

Quiescent HSC can be activated to divide by exposing such cells to Opn to promote uptake of an agent or transduction of genetic information. This aspect of the invention has important clinical implications, including improved transduction of genetic material into HSC via methods utilizing viral vectors (e.g., retroviral vector or lentiviral vectors), small interfering RNA molecules (RNAi), antisense, ribozymes, and the like for ex vivo manipulation of genetic expression, protein production and/or enzyme activation in the HSC population.

Quiescent HSC are activated in the presence of Opn or an active Opn fragments, including activation with Opn in the presence of thrombin, and cultured with an active agent or delivery vector. The actively dividing cells can promote genetic incorporation of genetic material, reproduction of genetic or viral elements within the cells, or activation of certain proteins during cell division. Such transformed/transduced HSC are useful for promoting gene expression and protein production for a number of therapeutic purposes, including correction of a genetic defect involving cells of the hematopoietic lineage or providing immunity to viral infection in progeny of the modified HSC (e.g., immunity to infection by HIV).

### EXAMPLES

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

### Example 1

### Analysis of the Spatial Distribution of HSC Using None Marrow Transplants in Non-Ablated Recipients

Transplantation into myeloablated recipients still remains the standard means by which patients are given a graft of HSC. However, the most appropriate method for analyzing the spatial distribution of cells within the bone marrow and the factors that regulate this process is one in which the HM has not been altered by preparative ablation. Using sex mismatched bone marrow transplants and detection of donor cells by in situ hybridization, the inventor previously reported the detection of transplanted HSC in the endosteal region (arbitrarily defined as 12 cells from the bone) six weeks post-transplant (Nilsson, S. K., et al., Blood, 1997. 89. p. 4013-4020). Subsequent studies in this laboratory confirm and extend these observations.

Recently, a novel approach has been developed using transplantation of fluorescently labeled (CFSE) cells, perfusion fixation and analysis of bone marrow sections to track individual cells lodging in non-ablated recipients. Transplants using different bone marrow sub-populations demonstrated that although the majority of cells entered the bone marrow from the central bone marrow vessels, their subsequent localization varied according to their phenotype. Populations enriched in HSC (Lin⁻Sca⁺Kit⁺ cells) exhibited selective migration and lodgment in the endosteal region while, in contrast, hematopoietic cells expressing surface markers associated with lineage commitment (designated Lin⁺) migrated away from the endosteal region, and demonstrated high selectivity for the central bone marrow region. Thus the distribution of transplanted hematopoietic cells within the bone marrow is not random and closely reflects that previously defined for related cell populations in steady state adult mouse BM (Lord, B. I., N. G. Testa, and J. H. Hendry, Blood, 1975. 46. p. 65-72). These data demonstrate for the first time that the discrete spatial localization of transplanted hematopoietic cells within the bone marrow appears to be the result of specific, hierarchically dependent patterns of migration that culminate in the retention of these populations at anatomically distinct sites. It is therefore proposed that the endosteal region of the bone marrow represents the site of HSC “niches”.

### Example 2

### The Interaction of HA and its Receptor CD44 in the Spatial Distribution of Transplanted HSC

Cell surface hyaluronic acid (HA) significantly affects the adhesion, motility and growth of a wide variety of cell types, both normal and neoplastic. Due to its multivalency (which allows cross bridging of multiple receptors on adjacent cells), the interaction of endogenous cell surface HA with its primary receptor, CD44, mediates aggregation of several cell types (Aruffo, A., et al., Cell, 1990. 61. p. 1303-1313). There are many examples of increased cell movement or invasion following either the exposure of cells to HA, or the ectopic expression of HA, and inhibition of cell movement occurs as a consequence of either HA degradation or the blocking of HA receptors (Turley, E. A., et al., Exp Cell Res, 1993. 207. p. 277-82).

Although HA has multiple receptors, the principal cell surface receptor is CD44 (Aruffo, A., et al., supra). There are many protein isoforms of CD44, with the most widely distributed being CD44H (H=haemopoietic). A murine model has been developed an utilized by the Inventor to analyze of the role of CD44 on bone marrow cells and within the hematopoietic microenvironment in the lodgment of engrafting HSC. In this model, recipients were created using lethally ablated CD44^{-/-} or C57B6 mice reconstituted with either normal C57B6 or CD44^{-/-} bone marrow for greater than 3 months. The spatial distribution of HSC isolated from either CD44^{-/-} or C57B6 mice was then analysed at short time-points post-transplant. Because stromal cells are not transplanted intravenously, this model allows the analysis of the effect of expression of CD44 by either bone marrow cells or the micronenvironment.

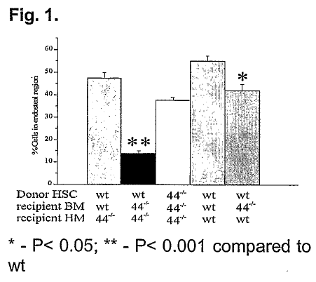

Analysis of engrafted HSC in this model demonstrated an important role for CD44 expressed on recipient bone marrow cells and within and lodgment within the endosteal region. Analysis of the spatial distribution of C57B6 HSC (Lin⁻Sca⁺Kit⁺) cells transplanted into non-ablated murine C57B6 recipients, that had previously been lethally irradiated and reconstituted with CD44^{-/-} normal bone marrow showed a significantly decreased proportion of donor cells in the endosteal region 15 hrs post-transplant (p<0.05) compared to a transplant of C57B6 HSC into C57B6 recipients, that had previously been lethally irradiated and reconstituted with C57B6 normal bone marrow (FIG. 1). In these recipients, the stromal-mediated microenvironment expresses CD44, and bone marrow cells are deficient in CD44. Transplanting C57B6 HSC (Lin⁻Sca⁺Kit⁺) into non-ablated murine CD44^{-/-} recipients, that had previously been lethally irradiated and reconstituted with CD44^{-/-} normal bone marrow 15 hrs post-transplant showed a totally random distribution of donor cells (FIG. 1); p<0.001 compared to wild-type. In these recipients, bone marrow cells and the microenvironment were both devoid of CD44. This suggests a functional role for the HA-CD44 interaction in the spatial distribution of engrafting HSC.

### Example 3

### CD44 Expression by Both HSC and the Hematopoietic Microenvironment is Crucial for HSC Potential in Vivo

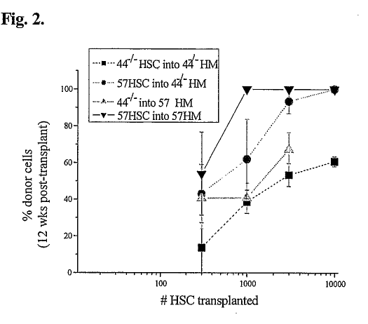

Analysis of HSC potential in a limiting dilution assay in vivo demonstrated a critical requirement for CD44 on both the donor HSC as well as within the recipient microenvironmental niche (FIG. 2). When CD44 was absent from the donor HSC, less than 50% chimerism was obtained 12 weeks following a transplant of 1000 HSC compared to 100% donor reconstitution following a transplant of equivalent numbers of wild type HSC. Furthermore, when wild type HSC were transplanted into a microenvironment devoid of CD44, significantly more HSC were required to obtain 100% donor reconstitution 12 weeks post-transplant compared to wild type HSC transplanted into a wild type HM. This suggests a functional role for CD44 in the regulation of HSC potential.

### Example 4

### The Physiological Role and Interactions of CD44 in HSC Lodgment

CD44 is ubiquitously expressed by cells within hematopoietic organs, with alternative splicing being tightly regulated and occurring only in particular cell types and activation states (Isacke, C. M. and H. Yarwood, Int J Biochem Cell Biol, 2002. 34. p. 718-21). CD44 has multiple ligands that mediate binding to a large range of cell types as well as the extracellular matrix proteins collagen, laminin and fibronectin (Wayner, E. A. and W. G. Carter, The Journal of Cell Biology, 1987. 105. p. 1873-1884; Faassen, A. E., et al., J Cell Biol, 1992. 116. p. 521-31; Jalkanen, S. and M. Jalkanen, J Cell Biol, 1992. 116. p. 817-25). Analysis of murine and human HSC demonstrated CD44H, CD44v6 and CD44v7 expression. In order to analyse the role of CD44 in HSC cell lodgment within the endosteum, an analysis of CD44 receptors with unique distribution to this region was undertaken. These investigations identified two potential candidates in the microenvironmental niche with well-documented interactions with CD44 in other cellular contexts, HA and Opn.

### Example 5

### The Physiological Role of Opn in HSC Lodgment

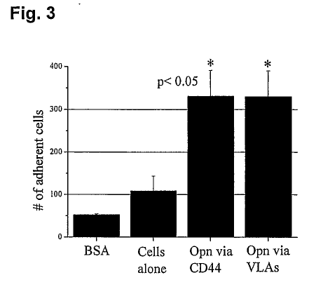

Labelling murine femoral sections with a specific anti-Opn antibody demonstrated a restricted expression of Opn to the endosteum. In addition, the inventor demonstrated that murine HSC bind to Opn through the β₁ integrins and CD44 (FIG. 3). This is the first demonstration of a specific interaction between HSC and Opn. When Opn is absent from the bone marrow microenvironmental niche, there is a significant (˜30%) reduction in the number of cells located at the endosteum 15 hrs post-transplant. Together, these data also suggest a functional role of the CD44-Opn interaction in the spatial distribution of engrafting HSC.

### Example 6

### The Role of Opn in HSC Regulation

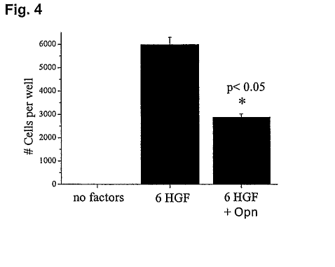

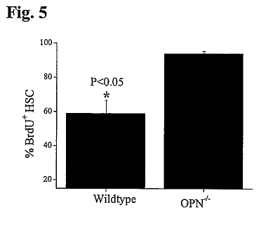

Recent experiments demonstrate that not only does HSC bind to Opn, but that these interactions inhibit proliferation of hematopoietic progenitors cells (FIG. 4). In these experiments, the addition of 2 μg/ml Opn to CD34⁺ cells inhibited overall cell proliferation by 50% after 4 days of culture. Despite the increase in overall proliferation, however, an analysis of the cycling history of HSC isolated from Opn^{-/-} and wild type mice following continuous oral bromodeoxyuridine (BrdU) administration for 4 weeks revealed a significantly faster cell cycle rate of HSC in Opn^{-/-} mice compared to wild type controls (FIG. 5). Together, these data suggest a key role for Opn in vivo in HSC regulation. The addition of Opn to HSC in culture has two key effects on cell proliferation: 1) Opn is inhibiting the total level of cell production of cells of the hematopoietic lineage; thus Opn is inhibiting proliferation and differentiation of the HSC; and 2) Opn is promoting the division of HSC into additional HSC, and thus enhancing the production of multipotential HSC in culture.

### Example 7

### CD44-Independent Activity of Opn in HSC-Microenvironmental Interactions

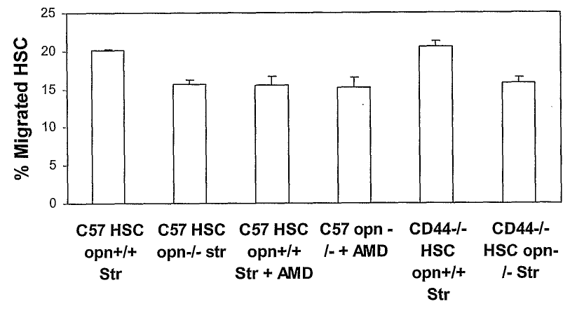

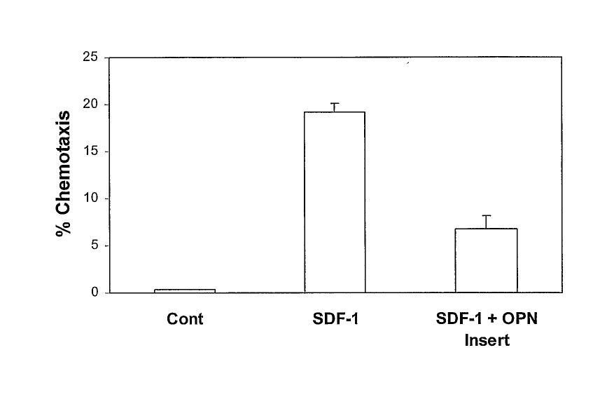

Additional data has shown that the activity of Opn is not completely dependent upon its interaction with CD44. Data demonstrating that the absence of Opn in the stroma inhibits the migration of HSC into the stroma is shown in FIG. 6. This suggests that Opn is involved with SDF-1 regulation, as normal migration of HSC into the stroma is largely due to SDF-1 (which is specifically inhibited by AMD). CD44 does not appear to be involved in this potential interaction of SDF-1 and Opn, as the results are the same in wild type and in a CD44 knock-out mouse. In addition, immobilized Opn has the ability to inhibit SDF-1 induced chemotaxis of HSC (FIG. 7).

This suggests that Opn is also having an effect independent from CD44 that impacts directly on lodgment of the HSC in the hematopoietic microenvironment.

While the present invention has been described with reference to specific embodiments, it should be understood by those skilled in the art that various changes may be made and equivalents may be substituted without departing from the true spirit and scope of the invention. In addition, many modifications may be made to adapt a particular situation, material, or process to the objective, spirit and scope of the present invention. All such modifications are intended to be within the scope of the invention.

1. A method for modulating ex vivo regulation, expansion, proliferation or differentiation of a multipotential stem cell population, said method comprising:
modulating exposure of the multipotential stem cell population to Opn and/or an active Opn fragment.

2. A method according to claim 1 for increasing ex vivo expansion of a multipotential stem cell population, said method comprising:
exposing the multipotential stem cell population to Opn and/or an active Opn fragment.

3. A method according to claim 2 wherein the Opn and/or an active Opn fragment is added to culture medium of the multipotential stem cell population culture.

4. A method according to claim 2 wherein the Opn and/or an active Opn fragment is immobilized on a culture device.

5. A method according to claim 4 wherein the culture device is selected from the group including a bead, culture flask or a bioreactor.

6. A method according to claim 2, further comprising:
adding thrombin.

7. A method according to claim 1 for increasing ex vivo proliferation and differentiation of a multipotential stem cell population, comprising:
decreasing or preventing exposure of the multipotential stem cell population to Opn and/or an active Opn fragment.

8. A method according to claim 7, comprising:
culturing the multipotential stem cell population in a culture medium without Opn and/or an active Opn fragment.

9. A method according to claim 7, comprising:
culturing the multipotential stem cell population in the presence of one or more inhibitors of Opn binding.

10. A multipotential cell population created by a method, comprising:
obtaining a multipotential stem cell population; and
culturing the multipotential stem cell population in a culture media including Opn and/or and active Opn fragment.

11. A differentiated cell population created by a method comprising:
obtaining a multipotential stem cell population; and
culturing the multipotential stem cell population in a culture media including one or more inhibitors of binding Opn and/or and active Opn fragment.

12. A cell culture medium including Opn and/or an active Opn fragment at a concentration which promotes expansion of a multipotential stem cell population.

13. A cell culture medium including an inhibitor of binding Opn and/or an active Opn fragment at a concentration which promotes differentiation of a multipotential stem cell population.

14. A method according to claim 1, wherein the multipotential stem cell is a haematopoietic stem cell.

15. A cell population according to claim 10 wherein the multipotential stem cell is a haematopoietic stem cell.

16. A culture medium according to claim 12 wherein the multipotential stem cell is a haematopoietic stem cell.

17. A cell population according to claim 11 wherein the multipotential stem cell is a haematopoietic stem cell.

18. A culture medium according to claim 13 wherein the multipotential stem cell is a haematopoietic stem cell.

19. A method according to claim 3 wherein the Opn and/or an active Opn fragment is immobilized on a culture device.

20. A method according to claim 8, comprising:
culturing the multipotential stem cell population in the presence of one or more inhibitors of Opn binding.
